SEQUENCE LISTING (8 sequences)

SEQ ID NO: 1 - DNA, 323 bp, artificial sequence
  This is a unique synthetic promoter is utilized , called SPc5-12
cggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcg

SEQ ID NO: 2 - DNA, 21 bp, artificial sequence
  This is a proximal serum response element (SRE) from skeletal alp ha-actin
gacacccaaatatggcgacgg

SEQ ID NO: 3 - DNA, 19 bp, artificial sequence
  A specific transcriptional regulatory region called MEF-1
ccaacacctgctgcctgcc

SEQ ID NO: 4 - DNA, 19 bp, artificial sequence
  A specific transcriptional regulatory region called MEF-1
cgctctaaaaataactccc

SEQ ID NO: 5 - DNA, 13 bp, artificial sequence
  A specific transcriptional regulatory region binding site called TEF-1
caccattcctcac

SEQ ID NO: 6 - DNA, 14 bp, artificial sequence
  A specific transcriptional regulatory region binding site called SP-1
ccgtccgccctcgg

SEQ ID NO: 7 - DNA, 4677 bp, artificial sequence
  This is an unidentified cloning vector for pSEAP2-Basic, with Accession  U89937
ggtaccgagctcttacgcgtgctagcccgggctcgagatctgcgatctaagtaagcttcgaatcgcgaattcgcccaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 8 - DNA, 5115 bp, artificial sequence
  This is an unidentified cloning vector for pSEAP2-Control, with Accession  U89938
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttcgaatcgcgaattcgcccaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

(19) United States
(12) Patent Application Publication
(10) Pub. No.: US 2003/0157717 A1
(43) Pub. Date: Aug. 21, 2003

(54) Linear DNA fragments for gene expression

(75) Inventor: Ruxandra Draghia-Akli, Houston, TX (US)

Correspondence Address: T. Ling Chwang, Jackson Walker L.L.P., Suite 600, 2435 N. Central Expressway, Richardson, TX, 75080, US

(73) Assignee: Baylor College of Medicine Texas Medical Center, Houston, TX (US)

(21) Appl. No.: 10/237,146

(22) Filed: Sep. 6, 2002

Related U.S. Application Data
(60) Provisional application No. 60/318,049, filed on Sep. 7, 2001.

(51) Int. Cl. C12N 15/85

(52) U.S. Cl. 435/455; 435/320.1

(57) ABSTRACT

Linear double-stranded DNA fragments containing a promoter, a nucleotide sequence, such as a transgene, preferably non-viral, and a 3′ untranslated region, are delivered to tissue of an animal by direct injection accompanied by electroporation. Long-term expression of the transgene results in prolonged availability of proteins, hormones, or enzymes that may be deficient in the mammal. In addition, the linear fragments increase the safety of the vectors for mammalian gene therapy by avoiding deleterious side effects.

### RELATED APPLICATIONS

This application claims priority to U.S. Provisional Patent Application, Serial No. 60/318,049, entitled “Linear DNA Fragments for Gene Expression,” filed on Sep. 7, 2001, the entire content of which is hereby incorporated by reference.

### BACKGROUND

One aspect of the current invention is a construct for plasmid mediated gene supplementation. The construct being a linear double-stranded nucleic acid expression plasmid substantially free from a viral backbone. The construct comprises a promoter; a nucleotide sequence of interest; and a 3′ untranslated region that are all operably linked. The in vivo expression of the nucleotide sequence of interest is regulated by the promoter. In a specific embodiment, the construct may comprise a residual linear plasmid backbone. The nucleotide sequence of interest in this invention encodes a hormone or an enzyme. A non-viral transgene that is used in the present invention comprises secreted alkaline phosphatase gene (“SEAP”) or a growth hormone releasing hormone (“GHRH”). The promoter of the construct comprises a tissue-specific promoter (e.g. SPc5-12) and the 3′ untranslated region comprises human growth hormone 3′ UTR, bovine growth hormone 3′ UTR, skeletal alpha actin 3′ UTR, or a SV40 polyadenylation signal. In a preferred embodiment, the present invention relates to a method for enhancing the synthesis of proteins and/or endogenous hormonal or enzymatic secretions in a subject through the delivery of the linear double stranded nucleotide expression construct that is substantially free from a viral backbone.

Plasmid mediated supplementation delivers nucleic acids to somatic tissue in a manner that can correct inborn or acquired deficiencies and imbalances. Nucleic acid vector-based drug delivery offers a number of advantages over the administration of recombinant proteins. These advantages include the conservation of native protein structure, improved biological activity, avoidance of systemic toxicities, and avoidance of infectious and toxic impurities. In addition, plasmid mediated gene supplementation allows for prolonged exposure to the protein in the therapeutic range, because the newly secreted protein is present continuously in the blood circulation.

The primary restriction of using recombinant protein is the limited availability of protein after each administration. Plasmid mediated gene supplementation using injectable DNA plasmid vectors overcomes this restriction, because a single injection into the patient's skeletal muscle permits physiologic expression for extensive periods of time (WO 99/05300 and WO 01/06988). Injection of the plasmid vectors promotes the production of enzymes and hormones in animals in a manner that more closely mimics the natural process. Furthermore, among the non-viral techniques for gene transfer in vivo, the direct injection of plasmid DNA into muscle tissue is simple, inexpensive, and safe.

In a plasmid based expression system, a non-viral gene vector may be composed of a synthetic gene delivery system in addition to the nucleic acid encoding a therapeutic gene product. In this way, the risks associated with the use of most viral vectors can be avoided. The non-viral expression vector products generally have low toxicity due to the use of “species-specific” components for gene delivery, which minimizes the risks of immunogenicity generally associated with viral vectors. Additionally, no integration of plasmid sequences into host chromosomes has been reported in vivo to date, thus, plasmid mediated gene supplementation should neither activate oncogenes nor inactivate tumor suppressor genes. Although not wanting to be bound by theory, as episomal systems residing outside the chromosomes, plasmids have defined pharmacokinetics and elimination profiles, leading to a finite duration of gene expression in target tissues.

Efforts have been made to enhance the delivery of plasmid DNA to cells by physical means including electroporation, sonoporation, and pressure. Although not wanting to be bound by theory, injection by electroporation involves the application of a pulsed electric field to create transient pores in the cellular membrane without causing permanent damage to the cell. It thereby allows for the introduction of exogenous molecules (Smith et al., 2000). By adjusting the electrical pulse generated by an electroporetic system, nucleic acid molecules can travel through passageways or pores in the cell that are created during the procedure. U.S. Pat. No. 5,704,908 describes an electroporation apparatus for delivering molecules to cells at a selected location within a cavity in the body of a patient. These pulse voltage injection devices are also described in U.S. Pat. Nos. 5,439,440 and 5,702,304, and PCT WO 96/12520, 96/12006, 95/19805, and 97/07826.

The electroporation technique has been used previously to transfect tumor cells after injection of plasmid DNA (Nishi et al., 1996; Rols et al., 1998), or to deliver the antitumoral drug bleomycin to cutaneous and subcutaneous tumors (Belehradek et al., 1994; Glass et al., 1996). Electroporation also has been used in rodents and other small animals (Mir et al., 1998; Muramatsu et al., 1998; Aihara et al., 1998). Advanced techniques of intramuscular injections of plasmid DNA followed by electroporation into skeletal muscle has been shown to lead to high levels of circulating growth hormone releasing hormone (GHRH), a hypothalamic hormone (Draghia-Akli et al., 1999; Draghia-Akli et al. 2002).

Other investigators have used linear fragments of DNA derived from adeno-associated vectors delivered by an intra-arterial high pressure hydrodynamic method to the liver and proved that these vectors can be efficacious and provide long term expression of a secreted protein (Chen et al., 2001). Mice injected with a linear DNA “expression cassette” (consisting of a promoter, a gene, and a 3′ UTR) encoding human alpha-1-antitrypsin (hAAT) expressed approximately 10 to 100-fold more serum hAAT than mice injected with closed circular DNA over the length of the study. However, these studies did not utilize electroporation, and the fragments retained adeno-associated viral backbone fragments. Thus, viral sequences were retained within the non-circular DNA fragments, and such practices give rise to several problems associated with viral backbone fragments (e.g. immunogenicity, insertional mutagenesis & toxicity problems).

The use of directly injectable DNA plasmid vectors has been limited in the past. The inefficient DNA uptake into muscle fibers after simple direct injection has led to relatively low expression levels (Prentice et al., 1994; Wells et al., 1997). In addition, the duration of the transgene expression has been short (Wolff et al., 1990; Danko et al., 1994). The most successful previous clinical applications have been confined to vaccines (Davis et al., 1994; Davis et al., 1997).

U.S. Pat. No. 4,956,288 is directed to methods for preparing recombinant host cells containing high copy number of a foreign DNA by electroporating a population of cells in the presence of the foreign DNA, culturing the cells, and killing the cells having a low copy number of the foreign DNA. Although there are references in the art directed to electroporation of eukaryotic cells with linear DNA (Neumann et al., 1982; McNally et al., 1988; Toneguzzo et al., 1988; Yorijufi and Mikawa, 1990; Aratani et al., 1992; Xie and Tsong, 1993; Nairn et al., 1993), these examples illustrate transfection into cell suspensions, cell cultures, and the like, and the transfected cells are not present in a somatic tissue.

Because viral vectors can induce an immunological response and have many inherent safety risks, e.g. insertional mutagenesis (Wang et al. 2002) and toxicity, lack of tissue specificity (Shi et al. 2002), and transcriptional silencing (Lund et al 1996), what is needed in the art, is a nucleic acid expression plasmid that is substantially free from the risks associated with viral vectors and can be delivered effectively and directly to somatic tissue. Of particular interest are linear double stranded nucleic acid expression constructs delivered to tissues through electroporation that lead to the long-term production of secreted hormones or enzymes.

### SUMMARY

One aspect of the present invention includes a double-stranded linear DNA expression construct substantially free from a viral backbone. The construct is utilized for the delivery of a nucleotide sequence, such as a transgene, to somatic tissues of an animal. It comprises a promoter (viral or non-viral), a nucleotide sequence, preferably a non-viral nucleotide sequence, and a 3′ end. The promoter, nucleotide sequence of interest, and 3′ UTR comprise the “expression cassette,” such that the nucleotide sequence can be expressed. Particular embodiments of the current invention, the promoter is tissue specific (e.g. muscle), synthetic, or specifically the SPc5-12 promoter. The SPc5-12 promoter preferably contains various combinations of muscle specific transcriptional regulatory regions such as SRE, MEF-1, MEF-2, TEF-1, and SP1. Non-viral transgenes that were used in specific embodiments of the present invention comprises secreted alkaline phosphatase gene (“SEAP”) or a growth hormone releasing hormone (“GHRH”). In a further specific embodiment, the 3′ end of the DNA fragment is an SV40 polyadenylation signal. Additionally, the linear double stranded nucleic acid expression construct was obtained through selective digestion of a circular DNA plasmid vector, such as pSP-SEAP2. The linear DNA expression construct was selectively cleaved to contain a bacterial replication origin, known as Uori. In another specific embodiment, the fragment also includes a packaging signal for the transgene, known as the Flori. In a further embodiment, the fragment contains the expression cassette and is delivered along with remaining fragments of the residual plasmid backbone that had been cut into pieces.

Another aspect the present invention includes a method of enhancing protein synthesis, hormonal or enzymatic secretions in cells of an animal comprising the steps of injecting an effective amount of a linear double-stranded expression construct directly into the targeted tissue of animals, then subjecting the cells to electroporation in order to facilitate the uptake of the construct. a double-stranded linear DNA expression construct substantially free from a viral backbone. The construct is utilized for the delivery of a nucleotide sequence, such as a transgene, to somatic tissues of an animal. It comprises a promoter (viral or non-viral), a nucleotide sequence, preferably a non-viral nucleotide sequence, and a 3′ end. The promoter, nucleotide sequence of interest, and 3′ UTR comprise the “expression cassette,” such that the nucleotide sequence can be expressed. Particular embodiments of the current invention, the promoter is tissue specific (e.g. muscle), synthetic, or specifically the SPc5-12 promoter. The SPc5-12 promoter preferably contains various combinations of muscle specific transcriptional regulatory regions such as SRE, MEF-1, MEF-2, TEF-1, and SP1. Non-viral transgenes that were used in specific embodiments of the present invention comprises secreted alkaline phosphatase gene (“SEAP”) or a growth hormone releasing hormone (“GHRH”). In a further specific embodiment, the 3′ end of the DNA fragment is an SV40 polyadenylation signal. Additionally, the linear double stranded nucleic acid expression construct was obtained through selective digestion of a circular DNA plasmid vector, such as pSP-SEAP2. The linear DNA expression construct was selectively cleaved to contain a bacterial replication origin, known as Uori. In another specific embodiment, the fragment also includes a packaging signal for the transgene, known as the Flori. In a further embodiment, the fragment contains the expression cassette and is delivered along with remaining fragments of the residual plasmid backbone that had been cut into pieces. Additionally, the linear double stranded nucleic acid expression construct was injected directly into the muscle tissue.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 illustrates the construct pSP-SEAP, which contains SPc5-12 synthetic promoter, a human SEAP gene, the SV40 polyadenylation signal (expression cassette), and a plasmid backbone with bacterial replication origin, Uori, an antibiotic resistance gene (ampicyllin), and a packaging origin for the SEAP gene, Flori. Different regions of the plasmid were cut using restriction enzymes (Sal I/Kpn I, Sal I/Ahd I, ApaL I/Kpn I, Sal I/Ahd I). Serum SEAP values in mice at 5, 11, 26 and 40 days post-injection (values in ng/mL; presented as average±standard error of the mean).

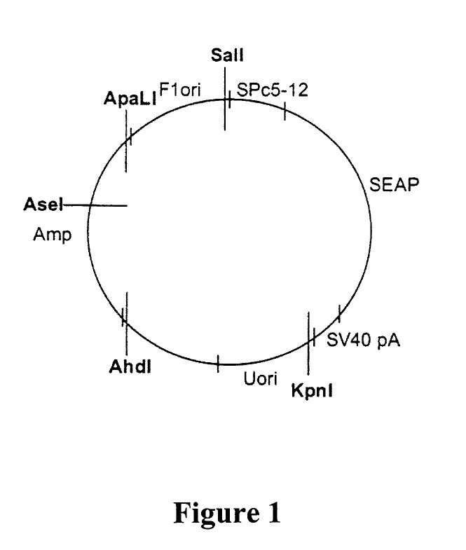

FIG. 2 demonstrates that groups of 5 severe combined immuno deficient (SCID) adult mice were injected with similar quantities of uncut circular pSP-SEAP, or fragments of pSP-SEAP as depicted in FIG. 1. Serum was analyzed for SEAP activity up to 76 days post-injection. SEAP activity was higher in mice injected with linear fragments containing either the expression cassette or the expression cassette and Fori.

### DETAILED DESCRIPTION OF THE INVENTION

I. Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

The term “cell-transfecting pulse” as used herein is defined as a transmission of a force which results in transfection of a vector, such as a linear DNA fragment, into a cell. In some embodiments, the force is from electricity, as in electroporation, or the force is from vascular pressure.

The term “coding region” as used herein refers to any portion of the DNA sequence that is transcribed into messenger RNA (mRNA) and then translated into a sequence of amino acids characteristic of a specific polypeptide.

The term “delivery” or “delivering” as used herein is defined as a means of introducing a material into a tissue, a subject, a cell or any recipient, by means of chemical or biological process, injection, mixing, electroporation, sonoporation, or combination thereof, either under or without pressure.

The term “DNA fragment” or “nucleic acid expression construct” as used herein refers to a substantially double stranded DNA molecule. Although the fragment may be generated by any standard molecular biology means known in the art, in some embodiments the DNA fragment or expression construct is generated by restriction digestion of a parent DNA molecule. The terms “expression vector,” “expression cassette,” or “expression plasmid” can also be used interchangeably. Although the parent molecule may be any standard molecular biology DNA reagent, in some embodiments the parent DNA molecule is a plasmid.

The terms “electrical pulse” and “electroporation” as used herein refer to the administration of an electrical current to a tissue or cell for the purpose of taking up a nucleic acid molecule into a cell. A skilled artisan recognizes that these terms are associated with the terms “pulsed electric field” “pulsed current device” and “pulse voltage device.” A skilled artisan recognizes that the amount and duration of the electrical pulse is dependent on the tissue, size, and overall health of the recipient subject, and furthermore knows how to determine such parameters empirically.

The term “encoded GHRH” as used herein is a biologically active polypeptide.

The term “growth hormone” (“GH”) as used herein is defined as a hormone that relates to growth and acts as a chemical messenger to exert its action on a target cell.

The term “growth hormone releasing hormone” (“GHRH”) as used herein is defined as a hormone that facilitates or stimulates release of growth hormone, and in a lesser extent other pituitary hormones, as prolactin.

The term “operatively linked” as used herein refers to elements or structures in a nucleic acid sequence that are linked by operative ability and not physical location. The elements or structures are capable of, or characterized by accomplishing a desired operation. It is recognized by one of ordinary skill in the art that it is not necessary for elements or structures in a nucleic acid sequence to be in a tandem or adjacent order to be operatively linked.

The term “plasmid” as used herein refers generally to a construction comprised of extra-chromosomal genetic material, usually of a circular duplex of DNA that can replicate independently of chromosomal DNA. Plasmids, or fragments thereof, may be used as vectors. Plasmids are double-stranded DNA molecule that occur or are derived from bacteria and (rarely) other microorganisms. However, mitochondrial and chloroplast DNA, yeast killer and other cases are commonly excluded.

The term “plasmid mediated gene supplementation” as used herein refers a method to allow a subject to have prolonged exposure to a therapeutic range of a therapeutic protein by utilizing a nucleic acid expression construct in vivo.

The term “pulse voltage device,” or “pulse voltage injection device” as used herein relates to an apparatus that is capable of causing or causes uptake of nucleic acid molecules into the cells of an organism by emitting a localized pulse of electricity to the cells. The cell membrane then destabilizes, forming passageways or pores. Conventional devices of this type are calibrated to allow one to select or adjust the desired voltage amplitude and the duration of the pulsed voltage. The primary importance of a pulse voltage device is the capability of the device to facilitate delivery of compositions of the invention, particularly linear DNA fragments, into the cells of the organism.

The term “plasmid backbone” as used herein refers to a sequence of DNA that typically contains a bacterial origin of replication, and a bacterial antibiotic selection gene, which are necessary for the specific growth of only the bacteria that are transformed with the proper plasmid. However, there are plasmids, called mini-circles, that lack both the antibiotic resistance gene and the origin of replication (Darquet et al., 1997; Darquet et al., 1999; Soubrier et al., 1999). The use of in vitro amplified expression plasmid DNA (i.e. non-viral expression systems) avoids the risks associated with viral vectors. The non-viral expression systems products generally have low toxicity due to the use of “species-specific” components for gene delivery, which minimizes the risks of immunogenicity generally associated with viral vectors. One aspect of the current invention is that the plasmid backbone does not contain viral nucleotide sequences.

The term “promoter” as used herein refers to a sequence of DNA that directs the transcription of a gene. A promoter may direct the transcription of a prokaryotic or eukaryotic gene. A promoter may be “inducible”, initiating transcription in response to an inducing agent or, in contrast, a promoter may be “constitutive”, whereby an inducing agent does not regulate the rate of transcription. A promoter may be regulated in a tissue-specific or tissue-preferred manner, such that it is only active in transcribing the operable linked coding region in a specific tissue type or types.

The term “replication element” as used herein comprises nucleic acid sequences that will lead to replication of a plasmid in a specified host. One skilled in the art of molecular biology will recognize that the replication element may include, but is not limited to a selectable marker gene promoter, a ribosomal binding site, a selectable marker gene sequence, and a origin of replication.

The term “residual linear plasmid backbone” as used herein comprises any fragment of the plasmid backbone that is left at the end of the process making the nucleic acid expression plasmid linear.

The term “subject” as used herein refers to any species of the animal kingdom. In preferred embodiments it refers more specifically to humans and animals used for: pets (e.g. cats, dogs, etc.); work (e.g. horses, cows, etc.); food (chicken, fish, lambs, pigs, etc); and all others known in the art.

The term “tissue” as used herein refers to a collection of similar cells and the intercellular substances surrounding them. A skilled artisan recognizes that a tissue is an aggregation of similarly specialized cells for the performance of a particular function. For the scope of the present invention, the term tissue does not refer to a cell line, a suspension of cells, or a culture of cells. In a specific embodiment, the tissue is electroporated in vivo. In another embodiment, the tissue is not a plant tissue. A skilled artisan recognizes that there are four basic tissues in the body: 1) epithelium; 2) connective tissues, including blood, bone, and cartilage; 3) muscle tissue; and 4) nerve tissue. In a specific embodiment, the methods and compositions are directed to transfer of linear DNA into a muscle tissue by electroporation.

The term “therapeutic element” as used herein comprises nucleic acid sequences that will lead to an in vivo expression of an encoded gene product. One skilled in the art of molecular biology will recognize that the therapeutic element may include, but is not limited to a promoter sequence, a transgene, a poly A sequence, or a 3′ or 5′ UTR.

The term “transfects” as used herein refers to introduction of a nucleic acid into a eukaryotic cell. In some embodiments, the cell is not a plant tissue or a yeast cell.

The term “vascular pressure pulse” refers to a pulse of pressure from a large volume of liquid to facilitate uptake of a vector into a cell. A skilled artisan recognizes that the amount and duration of the vascular pressure pulse is dependent on the tissue, size, and overall health of the recipient animal, and furthermore knows how to determine such parameters empirically.

The term “vector”0 as used herein refers to a construction comprised of genetic material designed to direct transformation of a targeted cell by delivering a nucleic acid sequence into that cell. A vector may contain multiple genetic elements positionally and sequentially oriented with other necessary elements such that an included nucleic acid cassette can be transcribed and when necessary translated in the transfected cells. These elements are operably linked. The term “expression vector” refers to a DNA plasmid that contains all of the information necessary to produce a recombinant protein in a heterologous cell.

The term “viral backbone” as used herein refers to a nucleic acid sequence that does not contain a promoter, a gene, and a 3′ poly A signal or an untranslated region, but contain elements including, but not limited at site-specific genomic integration Rep and inverted terminal repeats (“ITRs”) or the binding site for the tRNA primer for reverse transcription, or a nucleic acid sequence component that induces a viral immunogenicity response when inserted in vivo, allows integration, affects specificity and activity of tissue specific promoters, causes transcriptional silencing or poses safety risks to the subject.

II. The Present Invention

One aspect of the current invention is a construct for plasmid mediated gene supplementation. The construct being a linear double-stranded nucleic acid expression plasmid substantially free from a viral backbone. The construct comprises a promoter; a nucleotide sequence of interest; and a 3′ untranslated region that are all operably linked. The in vivo expression of the nucleotide sequence of interest is regulated by the promoter. In a specific embodiment, the construct may comprise a residual linear plasmid backbone. The nucleotide sequence of interest in this invention encodes a hormone or an enzyme, and in a specific embodiment includes growth hormone releasing hormone. Other hormones utilized as sequences of interest include: growth hormone, insulin, glucagon, adrenocorticotropic hormone, thyroid stimulating hormone, follicle-stimulating hormone, insulin growth factor I, insulin growth factor II, corticotropin-releasing hormone, parathyroid hormone, calcitonin, chorionic gonadotropin, luteinizing hormone, chorionic somatomammotropin, cholecystokinin, secretin, prolactin, oxytocin, vasopressin, angiotensin, melanocyte-stimulating hormone, somatostatin, thyrotropin-releasing hormone, gonadotropin-releasing hormone, or gastrin. Additionally, enzymes encoded as the nucleotide sequence of interest include a secreted embryonic alkaline phosphatase, glucuronidase, arylsulfatase, factor VIII, factor IX, or beta-galactosidase. Another embodiment of the current invention include the nucleotide sequence of interest encoding a cytokine (e.g. IL-2 or IL-7). The promoter of the construct comprises a tissue-specific promoter (e.g. SPc5-12). Furthermore, the 3′ untranslated region comprises human growth hormone 3′ UTR, bovine growth hormone 3′ UTR, skeletal alpha actin 3′ UTR, or a SV40 polyadenylation signal.

A second aspect of the current invention involves a method for increasing levels of a polypeptide in a subject. The method includes the steps of: delivering a linear double stranded nucleic acid expression construct, which is substantially free from a viral backbone, into a selected tissue, and applying a cell-transfecting pulse (e.g. an electric current) to the selected tissue. The polypeptide is encoded by a gene sequence on the linear double-stranded nucleic acid expression construct; and upon transfection of the construct to the cells, the levels of the encoded gene are elevated. In a specific embodiment, the linear double-stranded nucleic acid expression construct comprises a construct that is substantially free from a viral backbone having a promoter; a nucleotide sequence of interest; and a 3′ untranslated region that are all operably linked. The in vivo expression of the nucleotide sequence of interest is regulated by the promoter. In a specific embodiment, the construct may comprise a residual linear plasmid backbone. The nucleotide sequence of interest in this invention encodes a hormone or an enzyme, and in a specific embodiment includes growth hormone releasing hormone. Examples of other hormones or enzymes are also described herein. Another embodiment of the current invention include the nucleotide sequence of interest encoding a cytokine (e.g. IL-2 or IL-7). The promoter of the construct comprises a tissue-specific promoter (e.g. SPc5-12). Furthermore, the 3′ untranslated region comprises human growth hormone 3′ UTR, bovine growth hormone 3′ UTR, skeletal alpha actin 3′ UTR, or a SV40 polyadenylation signal.

An overall object of the present invention is to promote a long term expression of a nucleotide sequence, such as a transgene, encoding a protein, such as a hormone, an enzyme, or a cytokine, by the delivery of the nucleotide sequence to a somatic tissue of an animal, such as a mammal. A skilled artisan recognizes that, in a specific embodiment, the linear DNA fragments of the present invention contain only sequences that are “humanized”, or “mammalized”, and normally expressed in tissues (for instance GHRH gene, human growth hormone 3′ UTR, etc.) and not other sequences. Although not wanting to be bound by theory, given that the sequences of the nucleic acid expression construct are normally present, there is minimal or no risk for a significant immune response or for delivering oncogenic sequences to the animal upon administration of the fragments.

A further object of the present invention is to increase the uptake of DNA by the target cells by the use of particular delivery methods. Another object of the present invention is to deliver the DNA plasmid vectors directly to the somatic tissue. Still another object of the present invention is to use the vector of the present invention as a product supplement to an animal. A further object of the present invention is to avoid the risks associated with viral vectors in the delivery of a transgene.

One embodiment of the present invention is a linear double-stranded DNA fragment with a promoter, a nucleic acid sequence to be delivered to somatic tissue, and a 3′ untranslated region (“3′ end”), wherein the nucleotide sequence is expressed. In one embodiment, the nucleic acid sequence is a transgene. In a specific embodiment, the transgene is of non-viral origin.

A. Linear DNA Fragments

The linear DNA fragment can be obtained, for example, through selective cleavage of a circular DNA plasmid vector. One of skill in the art would be familiar with the methods of cleavage of circular DNA plasmid vector design, such as is described in Draghia-Akli et al. (1997), Li et al. (1999), and Draghia-Akli et al. (1999), all incorporated herein by reference. Other means of generating linear DNA fragments are known, such as by polymerase chain reaction, by mechanical shearing, by chemical shearing, and so forth.

In a specific embodiment, the pSP-SEAP2 vector (see Example 1) is utilized. This mammalian reporter vector contains the secreted alkaline phosphatase gene (SEAP), the transgene delivered in some specific embodiments. Lacking eukaryotic promoter and enhancer sequences, the pSP-SEAP2 vector has several characteristics that make it favorable for use. First, the sequences around the SEAP gene's ATP initiation codon generate a strong Kozak consensus translation initiation site. In addition, there is a multiple cloning site (MCS) upstream of the SEAP gene to allow for the insertion of promoters and to facilitate the selective digestion of the vector at particular points to create various linear DNA fragments.

The selective digestion of the circular vector by, for example, restriction enzymes and isolation of fragments allows for the preservation and removal of various sites on the vector. One such site preserved in a specific embodiment is the bacterial origin of replication site (Uori). This site, a specific nucleic acid sequence at which plasmid replication is initiated, assists in the propagation of a plasmid vector in the bacterial host cell for plasmid production. Another site preserved in a specific embodiment is the Flori site, which acts as a packaging origin for the SEAP gene. In another preferred embodiment, the remainder of the cleaved plasmid backbone is delivered along with the expression cassette. An additional plasmid feature that may be retained in the linear DNA fragments is the selectable marker, which aids in the identification of transformed cells, such as the gene conferring resistance to antibiotic.

Although not wanting to be bound by theory, there are multiple advantages of delivering DNA fragments in vivo from which the antibiotic resistance gene and/or the bacterial origin of replication have been removed. First, the antibiotic resistance gene could render the host organism resistant to that particular antibiotic. In addition, the ampicyllin gene contains multiple CpG motifs known to enhance the immune response in muscle cells (Stan et al., 2001). A less immuno-stimulatory vector can reduce the possibility of toxic responses and increase the therapeutic value of the vector (Yew et al., 2000). In addition, although undocumented for naked plasmid DNA, the possibility of plasmid replication in vivo is a possibility. The greatest transgene expression after plasmid DNA injection into skeletal muscle has been measured at 2-2.5 mm proximal to the site of injection (O'Hara et al., 2001). While some investigators are considering redesigned plasmids with conditional origins of replication, such as the pCOR plasmids (Soubrier et al., 1999), using the linear fragments that lack the bacterial origin of replication adds an extra step to creating safer plasmid mediated gene supplementation vectors.

B. Preferred Promoters

Where expression in a particular tissue is desired, strong non-tissue specific promoters, usually of viral origin, like CMV (cytomegalovirus promoter) may be replaced with tissue specific promoters within the vector.

However, in many embodiments of the present invention, tissue-specific expression is desired. For example, if the target tissue for gene expression is muscle, a synthetic muscle specific or an alpha-actin promoter may be employed. The avian skeletal alpha actin promoter is described in U.S. Pat. No. 5,298,422. Although not wanting to be bound by theory, several advantages may be gained through the use of tissue-specific promoters. In a particular tissue, such as muscle tissue, the use of muscle-specific promoters may increase the duration of expression. Tissue-specific promoters may be expected to decrease the potential for occult gene expression in non-target tissues. Additionally, tissue-specific promoters may provide the advantage of reduced expression in dendritic and other antigen presenting cells, thus avoiding immune responses to the expressed proteins. In certain circumstances, a low level of plasmid expression may also be desirable. In a combination plasmid system, it is also preferable to regulate the level of expression of a nucleotide sequence by inherent properties of the plasmid delivered rather than by attempting to variably titrate the dose of plasmid.

The MCS of most plasmids, such as the pSEAP2 vector, aids in the insertion of promoters. A preferred embodiment of the invention uses a muscle specific promoter made up of a series of muscle specific transcriptional regulatory regions having a novel configuration relative to those found in nature (PCT WO 99/02737). In one aspect of the present invention, a unique synthetic promoter is utilized, called SPc5-12 (Li et al., 1999). Although not wanting to be bound by theory, its transcriptional potency exceeds that of natural myogenic promoters. The SPc5-12 promoter (SEQ ID NO:1) has various synthetic orientations and combinations of muscle specific transcriptional regulatory regions, including proximal serum response element (SRE) from skeletal alpha-actin, multiple MEF-1 sites, multiple MEF-2 sites, TEF-1 binding sites, and SP-1, the sequences of which are set out below with the critical sequences underlined:

| | | | |
|---|---|---|---|
| SRE | 5′---- GACACCCAAATATGGCGACGG ----3′ | 21 mer | (SEQ ID NO:2) |
| MEF-1 | 5′---- CCAACACCTGCTGCCTGCC ----3′ | 19 mer | (SEQ ID NO:3) |
| MEF-2 | 5′---- CGCTCTAAAAATAACTCCC ----3′ | 19 mer | (SEQ ID NO:4) |
| TEF-1 | 5′---- CACCATTCCTCAC ----3′ | 13 mer | (SEQ ID NO:5) |
| SP-1 | 5′---- CCGTCCGCCCTCGG ----3′ | 14 mer | (SEQ ID NO:6) |

In one embodiment, a natural myogenic promoter is utilized, and a skilled artisan is aware how to obtain such promoter sequences from databases including the National Center for Biotechnology Information (NCBI) GenBank database or the NCBI PubMed site on the World Wide Web. A skilled artisan is aware that these World Wide Web sites may be utilized to obtain sequences or relevant literature related to the present invention.

C. Preferred 3′ Untranslated Regions

In further preferred embodiments, the 3′ UTR of the nucleic acid sequence is an SV40 polyadenylation signal. This signal is typically included in order to assure proper polyadenylation of the transcript. Other examples include human and bovine growth hormone 3′ UTR and skeletal alpha actin (3′ UTR).

D. Delivery of the Linear DNA Fragment to the Tissue

In additional specific embodiments, delivery of the linear DNA fragments is achieved by direct injection into the targeted somatic tissue. The type of injection device is not considered a limiting aspect of the present invention. A variety of means are known in the art to deliver the linear DNA fragments to the somatic tissue other than injection, such as by electroporation, gene gun, gold particles, and the like. A skilled artisan is aware that the same device may be used for both delivering the linear DNA fragments to the tissue and for transfecting, such as by electroporation, the fragments into cells. In some embodiments, the targeted tissue is muscle tissue.

E. Transfection of the Linear DNA Fragment into a Cell of the Tissue

Although not wanting to be bound by theory, following administration of the linear DNA fragments to the tissue, or concomitantly, the fragments are transfected into at least one cell of the tissue. The preferred delivery method utilizes electroporation immediately after injection. Applying a cell-transfecting pulse, such as by electricity or vascular pressure, to the targeted cells creates transient pores in the cell membrane to allow the DNA fragments to be taken up more efficiently. Once the fragments have been taken into, for example, the muscle fiber cells, the fragment then remains in the muscle fibers for, preferably, the life of the fibers. The linear fragments, or any other DNA fragments, remain in an episomal form. The delivered nucleic acid sequence, or transgene, is expressed, using the endogenous transcription machinery of the muscle fiber, and the transgene product is secreted from the fiber into the circulating blood to the target tissue. This ensures long-term production of secreted proteins, hormones, enzymes, or cytokines that may be naturally deficient in the target cells.

Effective transfer of a vector to a host cell in accordance with the present invention can be monitored by specialized assays which detect evidence of the transferred gene or expression of the gene within the host. For example, the presence of the SEAP gene product can be detected through a chemiluminescence assay of the test subject's blood.

The methods of the present invention are used to deliver therapeutic transgenes in a therapeutically effective amount. A therapeutically effective amount is the amount of the therapeutic transgene necessary for a therapeutic result in the cell and/or tissue. For example, fragments containing a growth hormone releasing hormone expression cassette are delivered to the skeletal muscle, GHRH is secreted and stimulates the synthesis and secretion of GH from the anterior pituitary. The product of the gene is easily detected in the serum by radio-immunoassay. The biological activity is analyzed by specific characteristics of the hormone or enzyme (i.e. increase weight for GH delivery). Similar methods are utilized for other therapeutic sequences.

III. Vectors

In some embodiments of the present invention, a linear DNA fragment is a vector. In some embodiments of the present invention, a linear DNA fragment is derived from another vector, such as a plasmid. The term “vector” is used to refer to a carrier nucleic acid molecule into which a nucleic acid sequence can be inserted for introduction into a cell wherein, in some embodiments, it can be replicated. A nucleic acid sequence can be native to the animal, or it can be “exogenous,” which means that it is foreign to the cell into which the vector is being introduced or that the sequence is homologous to a sequence in the cell but in a position within the host cell nucleic acid in which the sequence is ordinarily not found. Vectors include linear DNA fragments generated from plasmids, cosmids, viruses (bacteriophage, animal viruses, and plant viruses), and artificial chromosomes (e.g., YACs), although in a preferred embodiment the linear DNA fragment contains substantially no viral backbone. One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

F. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. Although not wanting to be bound by theory, the best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Although not wanting to be bound by theory, typically, these are located in the region 30-110 bp upstream of the start site, however, a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

Although not wanting to be bound by theory, the spacing between promoter elements frequently is flexible, so that promoter function is preserved when elements are inverted or moved relative to one another. In the TK promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicyllinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Although not wanting to be bound by theory, the control sequences that direct transcription and/or expression of sequences within non-nuclear organelles such as mitochondria, chloroplasts, and the like, can be employed.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, http://www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Tables 1 and 2 list non-limiting examples of elements/promoters that may be employed, in the context of the present invention, to regulate the expression of a RNA. Table 2 provides non-limiting examples of inducible elements, which are regions of a nucleic acid sequence that can be activated in response to a specific stimulus.

TABLE 1
Promoter and/or Enhancer

| Promoter/Enhancer | References |
|---|---|
| Immunoglobulin Heavy | Banerji et al., 1983; Gilles et al., 1983; |
| Chain | Grosschedl et al., 1985; Atchinson et al., |
| | 1986, 1987; Imler et al., 1987; Weinberger et |
| | al., 1984; Kiledjian et al., 1988; Porton et |
| | al.; 1990 |
| Immunoglobulin Light | Queen et al., 1983; Picard et al., 1984 |
| Chain | |
| T-Cell Receptor | Luria et al., 1987; Winoto et al., 1989; |
| | Redondo et al.; 1990 |
| HLA DQ a and/or DQ | Sullivan et al., 1987 |
| β | |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA- | Sherman et al., 1989 |
| Dra | |
| β-Actin | Kawamoto et al., 1988; Ng et al.; 1989 |
| Muscle Creatine | Jaynes et al., 1988; Horlick et al., 1989; |
| Kinase (MCK) | Johnson et al., 1989 |
| Prealbumin | Costa et al., 1988 |
| (Transthyretin) | |
| Elastase I | Omitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion | Hirsch et al., 1990 |
| Molecule (NCAM) | |
| α₁-Antitrypsin | Latimer et al., 1990 |
| H2B (TH2B) Histone | Hwang et al., 1990 |
| Mouse and/or Type I | Ripe et al., 1989 |
| Collagen | |
| Glucose-Regulated | Chang et al., 1989 |
| Proteins (GRP94 and | |
| GRP78) | |
| Rat Growth Hormone | Larsen et al., 1986 |
| Human Serum | Edbrooke et al., 1989 |
| Amyloid A (SAA) | |
| Troponin I (TN I) | Yutzey et al., 1989 |
| Platelet-Derived | Pech et al., 1989 |
| Growth Factor (PDGF) | |
| Duchenne Muscular | Kiamut et al., 1990 |
| Dystrophy | |
| SV40 | Banerji et al., 1981; Moreau et al., 1981; |
| | Sleigh et al., 1985; Firak et al., 1986; Herr |
| | et al., 1986; Imbra et al., 1986; Kadesch et |
| | al., 1986; Wang et al., 1986; Ondek et al., |
| | 1987; Kuhi et al., 1987; Schaffner et al., 1988 |
| Polyoma | Swartzendruber et al., 1975; Vasseur et al., |
| | 1980; Katinka et al., 1980, 1981; Tyndell et al., |
| | 1981; Dandolo et al., 1983; de Villiers et al., |
| | 1984; Hen et al., 1986; Satake et al., 1988; |
| | Campbell and/or Villarreal, 1988 |
| Retroviruses | Kriegler et al., 1982, 1983; Levinson et al., |
| | 1982; Kriegler et al., 1983, 1984a, b, 1988; |
| | Bosze et al., 1986; Miksicek et al., 1986; |
| | Celander et al., 1987; Thiesen et al., 1988; |
| | Celander et al., 1988; Choi et al., 1988; |
| | Reisman et al., 1989 |
| Papilloma Virus | Campo et al., 1983; Lusky et al., 1983; |
| | Spandidos and/or Wilkie, 1983; Spalholz et |
| | al., 1985; Lusky et al., 1986; Cripe et al., |
| | 1987; Gloss et al., 1987; Hirochika et al., |
| | 1987; Stephens et al., 1987 |
| Hepatitis B Virus | Bulla et al., 1986; Jameel et al., 1986; |
| | Shaul et al., 1987; Spandau et al., 1988; |
| | Vannice et al., 1988 |
| Human | Muesing et al., 1987; Hauber et al., 1988; |
| Immunodeficiency | Jakobovits et al., 1988; Feng et al., 1988; |
| Virus | Takebe et al., 1988; Rosen et al., 1988; |
| | Berkhout et al., 1989; Laspia et al., 1989; |
| | Sharp et al., 1989; Braddock et al., 1989 |
| Cytomegalovirus | Weber et al., 1984; Boshart et al., 1985; |
| (CMV) | Foecking et al., 1986 |
| Gibbon Ape Leukemia | Holbrook et al., 1987; Quinn et al., 1989 |
| Virus | |

TABLE 2
Inducible Elements

| Element | Inducer | References |
|---|---|---|
| MT II | Phorbol Ester (TFA) | Palmiter et al., 1982; Haslinger et |
| | Heavy metals | al., 1985; Searle et al., 1985; Stuart |
| | | et al., 1985; Imagawa et al., 1987, |
| | | Karin et al., 1987; Angel et al., |
| | | 1987b; McNeall et al., 1989 |
| MMTV | Glucocorticoids | Huang et al., 1981; Lee et al., |
| (mouse | | 1981; Majors et al., 1983; |
| mammary | | Chandler et al., 1983; Lee et al., |
| tumor virus) | | 1984; Ponta et al., 1985; Sakai et |
| | | al., 1988 |
| β-Interferon | Poly(rI)x | Tavernier et al., 1983 |
| | Poly(rc) | |
| Adenovirus | E1A | Imperiale et al., 1984 |
| 5 E2 | | |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX | Interferon, Newcastle | Hug et al., 1988 |
| Gene | Disease Virus | |
| GR P78 | A23187 | Resendez et al., 1988 |
| Gene | | |
| α-2- | IL-6 | Kunz et al., 1989 |
| Macroglobu- | | |
| lin | | |
| Vimentin | Serum | Rittling et al., 1989 |
| MHC Class | Interferon | Blanar et al., 1989 |
| I Gene H- | | |
| 2κb | | |
| HSP70 | E1A, SV40 Large T | Taylor et al., 1989, 1990a, 1990b |
| | Antigen | |
| Proliferin | Phorbol Ester-TPA | Mordacg et al., 1989 |
| Tumor | PMA | Hensel et al., 1989 |
| Necrosis | | |
| Factor α | | |
| Thyroid | Thyroid Hormone | Chatterjee et al., 1989 |
| Stimulating | | |
| Hormone α | | |
| Gene | | |

The identity of tissue-specific promoters or elements, as well as assays to characterize their activity, is well known to those of skill in the art. Non-limiting examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), and human platelet endothelial cell adhesion molecule-1 (Almendro et al., 1996).

In a preferred embodiment, a synthetic muscle promoter is utilized, such as SPc5-12 (Li et al., 1999), which contains a proximal serum response element (SRE) from skeletal α-actin, multiple MEF-2 sites, MEF-1 sites, and TEF-1 binding sites, and greatly exceeds the transcriptional potencies of natural myogenic promoters. The uniqueness of such a synthetic promoter is a significant improvement over, for instance, issued patents concerning a myogenic promoter and its use (e.g. U.S. Pat. No. 5,374,544) or systems for myogenic expression of a nucleic acid sequence (e.g. U.S. Pat. No. 5,298,422). In a preferred embodiment, the promoter utilized in the invention does not get shut off or reduced in activity significantly by endogenous cellular machinery or factors. Other elements, including trans-acting factor binding sites and enhancers may be used in accordance with this embodiment of the invention. In an alternative embodiment, a natural myogenic promoter is utilized, and a skilled artisan is aware how to obtain such promoter sequences from databases including the National Center for Biotechnology Information (NCBI) GenBank database or the NCBI PubMed site. A skilled artisan is aware that these databases may be utilized to obtain sequences or relevant literature related to the present invention.

G. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. Although not wanting to be bound by theory, the efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. Although not wanting to be bound by theory, by virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

H. Multiple Cloning Sites

Vectors can include a MCS, which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

I. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

J. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

K. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

L. Origins of Replication

In order to propagate a vector in a host cell, it may contain one or more origins of replication sites (often termed “ori”), which is a specific nucleic acid sequence at which replication is initiated. Alternatively an autonomously replicating sequence (“ARS”) can be employed if the host cell is yeast. In an embodiment of the invention, a residual plasmid backbone comprising an ori was described.

M. Selectable and Screenable Markers

In certain embodiments of the invention, cells containing a nucleic acid construct of the present invention can be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

N. Plasmid Vectors

In certain embodiments, a linear DNA fragment from a plasmid vector is contemplated for use to transfect a eukaryotic cell, particularly a mammalian cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicyllin and tetracycline resistance and thus provides easy means for identifying transformed cells. The pBR plasmid, or other microbial plasmid or phage must also contain, or be modified to contain, for example, promoters which can be used by the microbial organism for expression of its own proteins. A skilled artisan recognizes that any plasmid in the art may be modified for use in the methods of the present invention. In a specific embodiment, for example, a GHRH vector used for the therapeutical applications is derived from pBlueScript KS+ and has a kanamycin resistance gene.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

IV. Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding and other methods known in the art.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

V. Restriction Enzymes

In some embodiments of the present invention, a linear DNA fragment is generated by restriction enzyme digestion of a parent DNA molecule. Examples of restriction enzymes are provided in the following table.

| Name | Recognition Sequence |
|---|---|
| AatII | GACGTC |
| Acc65 I | GGTACC |
| Acc I | GTMKAC |
| Aci I | CCGC |
| Acl I | AACGTT |
| Afe I | AGCGCT |
| Afl II | CTTAAG |
| Afl III | ACRYGT |
| Age I | ACCGGT |
| Ahd I | GACNNNNNGTC |
| Alu I | AGCT |
| Alw I | GGATC |
| AlwN I | CAGNNNCTG |
| Apa I | GGGCCC |
| ApaL I | GTGCAC |
| Apo I | RAATTY |
| Asc I | GGCGCGCC |
| Ase I | ATTAAT |
| Ava I | CYCGRG |
| Ava II | GGWCC |
| Avr II | CCTAGG |
| Bae I | NACNNNNGTAPyCN |
| BamH I | GGATCC |
| Ban I | GGYRCC |
| Ban II | GRGCYC |
| Bbs I | GAAGAC |
| Bbv I | GCAGC |
| BbvC I | CCTCAGC |
| Bcg I | CGANNNNNNTGC |
| BciV I | GTATCC |
| Bcl I | TGATCA |
| Bfa I | CTAG |
| Bgl I | GCCNNNNNGGC |
| Bgl II | AGATCT |
| Blp I | GCTNAGC |
| Bmr I | ACTGGG |
| Bpm I | CTGGAG |
| BsaA I | YACGTR |
| BsaB I | GATNNNNATC |
| BsaH I | GRCGYC |
| Bsa I | GGTCTC |
| BsaJ I | CCNNGG |
| BsaW I | WCCGGW |
| BseR I | GAGGAG |
| Bsg I | GTGCAG |
| BsiE I | CGRYCG |
| BsiHKA I | GWGCWC |
| BsiW I | CGTACG |
| Bsl I | CCNNNNNNNGG |
| BsmA I | GTCTC |
| BsmB I | CGTCTC |
| BsmF I | GGGAC |
| Bsm I | GAATGC |
| BsoB I | CYCGRG |
| Bsp1286 I | GDGCHC |
| BspD I | ATCGAT |
| BspE I | TCCGGA |
| BspH I | TCATGA |
| BspM I | ACCTGC |
| BsrB I | CCGCTC |
| BsrD I | GCAATG |
| BsrF I | RCCGGY |
| BsrG I | TGTACA |
| Bsr I | ACTGG |
| BssH II | GCGCGC |
| BssK I | CCNGG |
| Bst4C I | ACNGT |
| BssS I | CACGAG |
| BstAP I | GCANNNNNTGC |
| BstB I | TTCGAA |
| BstE II | GGTNACC |
| BstF5 I | GGATGNN |
| BstN I | CCWGG |
| BstU I | CGCG |
| BstX I | CCANNNNNNTGG |
| BstY I | RGATCY |
| BstZ17 I | GTATAC |
| Bsu36 I | CCTNAGG |
| Btg I | CCPuPyGG |
| Btr I | CACGTG |
| Cac8 I | GCNNGC |
| Cla I | ATCGAT |
| Dde I | CTNAG |
| Dpn I | GATC |
| Dpn II | GATC |
| Dra I | TTTAAA |
| Dra III | CACNNNGTG |
| Drd I | GACNNNNNNGTC |
| Eae I | YGGCCR |
| Eag I | CGGCCG |
| Ear I | CTCTTC |
| Eci I | GGCGGA |
| EcoN I | CCTNNNNNAGG |
| EcoO109 I | RGGNCCY |
| EcoR I | GAATTC |
| EcoR V | GATATC |
| Fau I | CCCGCNNNN |
| Fnu4H I | GCNGC |
| Fok I | GGATG |
| Ese I | GGCCGGCC |
| Fsp I | TGCGCA |
| Hae II | RGCGCY |
| Hae III | GGCC |
| Hga I | GACGC |
| Hha I | GCGC |
| Hinc II | GTYRAC |
| Hind III | AAGCTT |
| Hinf I | GANTC |
| HinP1 I | GCGC |
| Hpa I | GTTAAC |
| Hpa II | CCGG |
| Hph I | GGTGA |
| Kas I | GGCGCC |
| Kpn I | GGTACC |
| Mbo I | GATC |
| Mbo II | GAAGA |
| Mfe I | CAATTG |
| Mlu I | ACGCGT |
| Mly I | GAGTCNNNNN |
| Mnl I | CCTC |
| Msc I | TGGCCA |
| Mse I | TTAA |
| Msl I | CAYNNNNRTG |
| MspA1 I | CMGCKG |
| Msp I | CCGG |
| Mwo I | GCNNNNNNNGC |
| Nac I | GCCGGC |
| Nar I | GGCGCC |
| Nci I | CCSGG |
| Nco I | CCATGG |
| Nde I | CATATG |
| NgoMI V | GCCGGC |
| Nhe I | GCTAGC |
| Nla III | CATG |
| Nla IV | GGNNCC |
| Not I | GCGGCCGC |
| Nru I | TCGCGA |
| Nsi I | ATGCAT |
| Nsp I | RCATGY |
| Pac I | TTAATTAA |
| PaeR7 I | CTCGAG |
| Pci I | ACATGT |
| PflF I | GACNNNGTC |
| PflM I | CCANNNNNTGG |
| PleI | GAGTC |
| Pme I | GTTTAAAC |
| Pml I | CACGTG |
| PpuM I | RGGWCCY |
| PshA I | GACNNNNGTC |
| Psi I | TTATAA |
| PspG I | CCWGG |
| PspOM I | GGGCCC |
| Pst I | CTGCAG |
| Pvu I | CGATCG |
| Pvu II | CAGCTG |
| Rsa I | GTAC |
| Rsr II | CGGWCCG |
| Sac I | GAGCTC |
| Sac II | CCGCGG |
| Sal I | GTCGAC |
| Sap I | GCTCTTC |
| Sau3A I | GATC |
| Sau96 I | GGNCC |
| Sbf I | CCTGCAGG |
| Sca I | AGTACT |
| SerF I | CCNGG |
| SexA I | ACCWGGT |
| SfaN I | GCATC |
| Sfc I | CTRYAG |
| Sfi I | GGCCNNNNNGGCC |
| Sfo I | GGCGCC |
| SgrA I | CRCCGGYG |
| Sma I | CCCGGG |
| Sml I | CTYRAG |
| SnaB I | TACGTA |
| Spe I | ACTAGT |
| Sph I | GCATGC |
| Ssp I | AATATT |
| Stu I | AGGCCT |
| Sty I | CCWWGG |
| Swa I | ATTTAAAT |
| Taq I | TCGA |
| Tfi I | GAWTC |
| Tli I | CTCGAG |
| Tse I | GCWGC |
| Tsp45 I | GTSAC |
| Tsp509 | I AATT |
| TspR I | CAGTG |
| Tthl11 I | GACNNNGTC |
| Xba I | TCTAGA |
| Xcm I | CCANNNNNNNNNTGG |
| Xho I | CTCGAG |
| Xma I | CCCGGG |
| Xmn I | GAANNNNTTC |

The term “restriction enzyme digestion” of DNA as used herein refers to catalytic cleavage of the DNA with an enzyme that acts only at certain locations in the DNA. Such enzymes are called restriction endonucleases, and the sites for which each is specific is called a restriction site. The various restriction enzymes used herein are commercially available and their reaction conditions, cofactors, and other requirements as established by the enzyme suppliers are used. Restriction enzymes commonly are designated by abbreviations composed of a capital letter followed by other letters representing the microorganism from which each restriction enzyme originally was obtained and then a number designating the particular enzyme. In general, about 1 μg of plasmid or DNA fragment is used with about 1-2 units of enzyme in about 20 μl of buffered solution. Appropriate buffers and substrate amounts for particular restriction enzymes are specified by the manufacturer. Incubation of about 1 hour at 37° C. is ordinarily used, but may vary in accordance with the supplier's instructions. After incubation, protein or polypeptide is removed by extraction with phenol and chloroform, and the digested nucleic acid is recovered from the aqueous fraction by precipitation with ethanol. Digestion with a restriction enzyme may be followed with bacterial alkaline phosphatase hydrolysis of the terminal 5 phosphates to prevent the two restriction cleaved ends of a DNA fragment from “circularizing” or forming a closed loop that would impede insertion of another DNA fragment at the restriction site. Unless otherwise stated, digestion of plasmids is not followed by 5′ terminal dephosphorylation. Procedures and reagents for dephosphorylation are conventional as described in Sambrook et al. (1989).

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the invention.

### Example 1

### Vector Digestion and Fragment Isolation

The pSEAP2 mammalian reporter vector, containing the non-viral, human SEAP gene (Clontech Laboratories, Inc., Palo Alto, Calif.) was used in these studies. In this particular case, the strong muscle specific synthetic promoter SPc5-12 was inserted into the pSEAP2 basic vector, to create a pSP-SEAP vector. The SEAP coding sequence is followed by the SV40 late polyadenylation signal to ensure proper, efficient processing of the transcript. The vector backbone also provides an f1 origin for single-stranded DNA production, a pUC19 (prokaryotic) bacterial origin of replication, and an ampicillin (prokaryotic) resistance gene for propagation and selection in E. coli. The vector also has a MCS with digestion sites for restriction enzymes: pSEAP2-Basic 5′-Asp718 I, Kpn I, Mlu I, Nhe I, Srf I, Xho I, BglII, Hind III, BstB I, Nru I, and EcoR I -3′. (GenBank Accession Numbers: pSEAP 2-Basic (SEQ ID NO:7; U89937); pSEAP2-Control (SEQ ID NO:8; U89938).

The vector pSP-SEAP was amplified into DH5α competent cells and the plasmid purification was achieved using a Qiagen Endotoxin Free Giga kit (Qiagen; Valencia, Calif.). At the end of the purification process, the plasmid was resuspended in water and stored at −80° C. until usage.

Several linear plasmid DNA fragments were generated by specific restriction enzyme digestion of the circular DNA, followed by electrophoretic gel migration, separation of fragments, isolation of fragments, and linear plasmid DNA gel extraction using the QIAquik DNA Cleanup system (Qiagen, Valencia, Calif.). DNA concentration was determined first by spectroscopy. The fragments were stored in water at −80° C. until usage. Samples of each fragment were migrated onto a 1% agarose gel, and the correct dimension and concentration was confirmed.

### Example 2

### Linear DNA Fragments

Four different digestions of the pSP-SEAP vector were performed, with four different linear DNA fragments isolated and used. The first digestion used the restriction enzymes Kpn I and Sal I. The fragment remaining after isolation contained only the SPc5-12 promoter, the SEAP gene, and the SV40 polyadenylation signal. These three regions, a promoter, a nucleotide sequence of interest, and a polyA signal, together are known as the “expression cassette.” The second digestion utilized the restriction enzymes Kpn I and Ahd I and resulted in a DNA fragment containing the expression cassette and the bacterial origin of replication. The restriction enzymes ApaL I and Sal I were used in the third digestion. The resulting DNA fragment contained the expression cassette and the f1 origin. The final digestion used three restriction enzymes, Kpn I, Sal I, and Ase I, and resulted in a fragment containing the expression cassette, along with the plasmid backbone cut into two pieces. A skilled artisan is aware how to remove undesirable fragments from desirable fragments, such as by electrophoresis.

### Example 3

### Fragment Delivery and Animal Studies

The SEAP gene is an immunogenic protein in normal, adult mice. In order to avoid an immune reaction against the transgene and to enable a study of the long-term expression of the different non-circular DNA fragments, severe combined immuno-deficient (SCID) mice were used as the experimental model. The SCID male mice were housed and cared for under environmental conditions of 10 hours of light, followed by 14 hours of darkness. The mice were maintained in accordance with NIH Guide, USDA and Animal Welfare Act guidelines, and the protocol was approved by the Institutional Animal Care and Use Committee. On day 0, the mice (n=5 per group) were weighed. Then, their left tibialis anterior muscles were injected with 8 micrograms of DNA diluted in 25 μL sterile deionized water. Of the six tested groups, one received uncut, circular DNA, four received one particular type of the fragments listed above, and one control group received an injection of PBS. The injection was followed by electroporation, using external caliper electrodes and standard conditions of 6 pulses, 60 milliseconds/pulse, 100 V/cm, (Draghia-Akli et al., 1999). A BTX T820 generator (BTX, division of Genetronics Inc., Calif.) was used to deliver square wave pulses in all experiments.

### Example 4

### Measuring Expression of SEAP

Blood samples from the mice were collected starting on the fifth day after injection. The collected serum was subjected to a chemiluminescent assay to detect the presence of the SEAP gene.

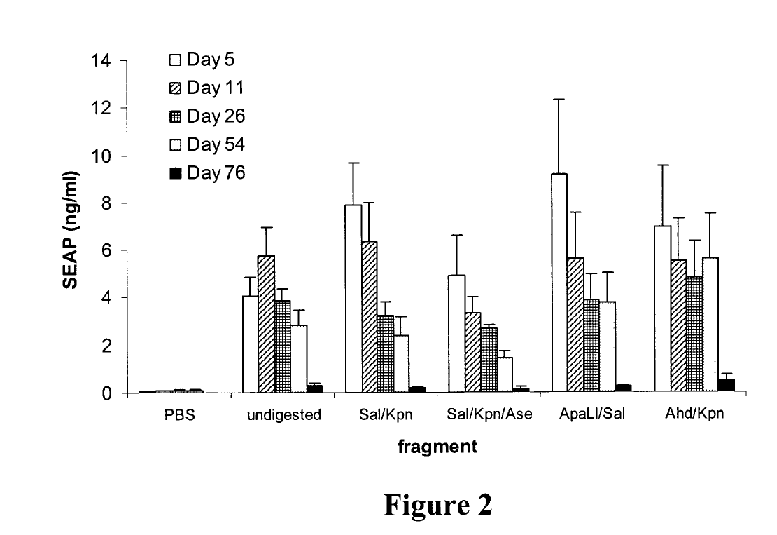

FIG. 2 and Table 3 represent serum SEAP values in mice at 5, 11, 26, 54, and 76 days post-injection (values in ng/mL; presented as average±standard error of the mean (+/−SE)).

| | Day 5 | Day 11 | Day 26 | Day 54 | Day 76 |
|---|---|---|---|---|---|
| SEAP (ng/ml) | | | | | |
| PBS | 0.040 | 0.100 | 0.100 | 0.090 | 0.020 |
| undigested | 4.090 | 5.780 | 3.860 | 2.830 | 0.310 |
| Sal/Kpn | 7.880 | 6.360 | 3.240 | 2.400 | 0.200 |
| Sal/Kpn/Ase | 4.910 | 3.320 | 2.660 | 1.420 | 0.170 |
| ApaLl/Sal | 9.200 | 5.620 | 3.850 | 3.770 | 0.230 |
| Ahd/Kpn | 6.960 | 5.520 | 4.810 | 5.620 | 0.470 |
| (+/−) SE | | | | | |
| PBS | 0.004 | 0.002 | 0.059 | 0.054 | 0.006 |
| undigested | 0.763 | 1.159 | 0.498 | 0.659 | 0.088 |
| Sal/Kpn | 1.794 | 1.620 | 0.594 | 0.771 | 0.064 |
| Sal/Kpn/Ase | 1.690 | 0.684 | 0.183 | 0.332 | 0.057 |
| ApaLl/Sal | 3.120 | 1.918 | 1.136 | 1.233 | 0.090 |
| Ahd/Kpn | 2.549 | 1.780 | 1.541 | 1.860 | 0.268 |

It should be noted that expression from all linear plasmid DNA fragments delivered to the skeletal muscle by electroporation gave higher or equal expression compared to the circular plasmid DNA on day 5. The fragment ApaL I/Sal I containing the expression cassette and the f1 origin, without the antibiotic resistance gene, gave high expression past day 54.

Delivering to a mammal a plasmid fragment that lacks components of the antibiotic gene is beneficial in that there is minimal risk of introducing an antibiotic resistance gene to the mammal. The bacterial origin of replication is essential for bacterial proliferation, and fragments that do not contain this fragment are incapable of replicating in vivo. Thus, in preferred embodiment, the fragment lacking in the bacterial origin of replication gives extra protection for the plasmid mediated gene supplementation applications.

One skilled in the art readily appreciates that the patent invention is well adapted to carry out the objectives and obtain the ends and advantages mentioned as well as those inherent therein. Methods, procedures, techniques, plasmids, linear fragments, and kits described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

### REFERENCES CITED

All patents and publications mentioned in the specification are indicative of the levels of those skilled in the art to which the invention pertains. All patents and publications are herein incorporated by reference to the same extent as if each individual publication was specifically and individually indicated to be incorporated by reference.

### U.S. Patent Documents

U.S. Pat. No. 5,935,819 titled “Process for producing a pharmaceutical preparation of PDGF-AB” issued on Aug. 10, 1999 with Eichner et al., listed as inventors.

U.S. Pat. No. 5,928,906 titled “Process for direct sequencing during template amplification” issued on Jul. 27, 1999 with Koster et al., listed as inventors.

U.S. Pat. No. 5,925,565 titled “Internal ribosome entry site, vector containing it and therapeutic use” issued on Jul. 20, 1999 with Berlioz et al., listed as inventors.

U.S. Pat. No. 5,704,908 issued on Jan. 6, 1998 with Gunter A. Hofmann and Lois J. Crandell listed as inventors.

U.S. Pat. No. 5,702,384 issued on Dec. 30, 1997 with Koichi Umeyama, Tadahiko Ogasawara, Kenji Yoshino, Katsushi Watanabe, and Koji Koda listed as inventors.

U.S. Pat. No. 5,439,440 issued on Aug. 8, 1995 with Gunter A. Hofmann listed as inventor.

U.S. Pat. No. 5,384,253 titled “Genetic transformation of maize cells by electroporation of cells pretreated with pectin degrading enzymes” issued on Jan. 24, 1995 with Krzyzek et al., listed as inventors.

U.S. Pat. No. 5,374,544 titled “Mutated skeletal actin promoter” issued on Dec. 20, 1994 with Schwartz et al., listed as inventors.

U.S. Pat. No. 5,298,422 titled “Myogenic vector systems” issued on Mar. 29, 1994 with Schwartz et al., listed as inventors.

U.S. Pat. No. 4,683,202 titled “Process for amplifying nucleic acid sequences” issued on Jul. 28, 1987 with Mullis et al., listed as inventors.

U.S. Pat. No. 4,956,288 issued on Sep. 11, 1990 with James G. Barsoum listed as inventor.

Patent Cooperation Treaty No. WO 99/05300 published on Feb. 4, 1999 and titled “GHRH Expression system and methods of use,” with Schwartz et al., listed as inventors.

Patent Cooperation Treaty No. WO 01/06988 published on Feb. 1, 2001 and titled “Super-active porcine growth hormone releasing hormone analog,” with Schwartz et al., listed as inventors.

Patent Cooperation Treaty No. WO 96/12520 published on May 2, 1996 and titled “Electroporetic Gene and drug Therapy by induced electric fields,” with Hoffman listed as inventor.

Patent Cooperation Treaty No. WO 96/12006 published on Apr. 25, 1996 and titled “Flow through electroporation apparatus and method,” with Hoffman listed as inventor.

Patent Cooperation Treaty No. WO 97/07826 published on Mar. 6, 1997 and titled “In vivo electroporation of cells,” with Nicolau et al., listed as inventors.

Patent Cooperation Treaty No. WO 95/19805 published on Jul. 27, 1995 and titled “Electroporation and iontophoresis apparatus and method for insertion of drugs and genes into cells,” with Hoffman et al., listed as inventors.

### Publications

Aihara, H., Miyazaki, J. (1998) Gene transfer into muscle by electroporation in vivo. Nat. Biotechnol. 16, 867-870

Almendro N, Bellon T, Rius C, Lastres P, et al. Cloning of the human platelet endothelial cell adhesion molecule-1 promoter and its tissue-specific expression. Structural and functional characterization. J Immunol Dec. 15, 1996; 157(12):5411-21.

Angel et al., Cell, 49:729, 1987b.

Angel et al., Mol. Cell. Biol., 7:2256, 1987a.

Aratani, Y., Okazaki, R., Koyama, H. (1992) End extension repair of introduced targeting vectors mediated by homologous recombination in mammalian cells. Nucl Acids Res. 20 (18):4795-4801

Atchison and Perry, Cell, 46:253, 1986.

Atchison and Perry, Cell, 48:121, 1987.

Banerji et al., Cell, 27:299, 1981.

Banerji et al., Cell, 35:729, 1983.

Belehradek, J., Orlowski, S., Ramirez, L. H., Pron, G., Poddevin, B., Mir, L. M. (1994) Electropermeabilization of cells in tissues assessed by the qualitative and quantitative electroloading of bleomycin. Biochim.Biophys.Acta 1190, 155-163

Berkhout et al., Cell, 59:273, 1989.

Blanar et al., EMBO J., 8:1139, 1989.

Bodine and Ley, EMBO J., 6:2997, 1987.

Boshart et al., Cell, 41:521, 1985.

Bosze et al., EMBO J., 5:1615, 1986.

Braddock et al., Cell, 58:269, 1989.

Bulla and Siddiqui, J. Virol., 62:1437, 1986.

Campbell and Villarreal, Mol. Cell. Biol., 8:1993, 1988.

Campere and Tilghman, Genes and Dev., 3:537, 1989.

Campo et al., Nature, 303:77, 1983.

Celander and Haseltine, J. Virology, 61:269, 1987.

Celander et al., J. Virology, 62:1314, 1988.

Chandler et al., Cell, 33:489, 1983.

Chang et al., Mol. Cell. Biol., 9:2153, 1989.

Chatterjee et al., Proc. Nat'l Acad. Sci. USA., 86:9114, 1989.

Chen, Z. Y., Yant, S. R., He, C. Y., Meuse, L., Shen, S., Kay, M. A. (2001) Linear DNAs concatemerize in vivo and result in sustained transgene expression in mouse liver. Mol.Ther. 3, 403-410

Choi et al., Cell, 53:519, 1988.

Cohen et al., “A Repetitive Sequence Element 3′ of the human c-Ha-ras1 Gene Has Enhancer Activity,” J. Cell. Physiol., 5:75, 1987.

Costa et al., Mol. Cell. Biol., 8:81, 1988.

Cripe et al., EMBO J., 6:3745, 1987.

Culotta and Hamer, Mol. Cell. Biol., 9:1376, 1989.

Dandolo et al., J. Virology, 47:55, 1983.

Danko, I., Fritz, J. D., Jiao, S., Hogan, K., Latendresse, J. S., Wolff, J. A. (1994) Pharmacological enhancement of in vivo foreign gene expression in muscle. Gene Therapy 1, 114-121

Davis, H. L. (1997) Plasmid DNA expression systems for the purpose of immunization [Review]. Current Opinion in Biotechnology 8, 635-640

Davis, H. L., Michel, M. L., Mancini, M., Schleef, M., Whalen, R. G. (1994) Direct gene transfer in skeletal muscle: plasmid DNA-based immunization against the hepatitis B virus surface antigen. Vaccine 12, 1503-1509

De Villiers et al., Nature, 312:242, 1984.

Deschamps et al., Science, 230:1174, 1985.

Draghia-Akli, R., Malone, P. B.; Hill, L. A.; Ellis, K. M.; Schwartz, R. J.; Nordstrom, J. L. (2002) Enhanced animal growth via ligand-regulated GHRH myogenic-injectable vectors. The FASEB Journal 16 (3): 426-428

Draghia-Akli, R., Fiorotto, M. L., Hill, L. A., Malone, P. B., Deaver, D. R., Schwartz, R. J. (1999) Myogenic expression of an injectable protease-resistant growth hormone-releasing hormone augments long-term growth in pigs. Nat. Biotechnol. 17, 1179-1183

Draghia-Akli, R., Li, X. G., Schwartz, R. J. (1997) Enhanced growth by ectopic expression of growth hormone releasing hormone using an injectable myogenic vector. nature biotechnology 15, 1285-1289

Edlund et al., Science, 230:912, 1985.

Feng and Holland, Nature, 334:6178, 1988.

Firak and Subramanian, Mol. Cell. Biol., 6:3667, 1986.

Foecking and Hofstetter, “Powerful and/or Versatile Enhancer-Promoter Unit for [mammalian, plant, fungus, bacteria?] Expression Vectors,” Gene, 45:101, 1986.

Fujita et al., Cell, 49:357, 1987.

Gilles et al., Cell, 33:717, 1983.

Glass, L. F., Pepine, M. L., Fenske, N. A., Jaroszeski, M., Reintgen, D. S., Heller, R. (1996) Bleomycin-mediated electrochemotherapy of metastatic melanoma. Arch. Dermatol. 132, 1353-1357

Gloss et al., EMBO J., 6:3735, 1987.

Godbout et al., Mol. Cell. Biol., 8:1169, 1988.

Goodbourn and Maniatis, Proc. Nat'l Acad. Sci. USA, 85:1447, 1988.

Goodbourn et al., Cell, 45:601, 1986.

Greene et al., Immunology Today, 10:272, 1989.

Grosschedl and Baltimore, Cell, 41:885, 1985.

Haslinger and Karin, Proc. Nat'l Acad. Sci. USA., 82:8572, 1985.

Hauber and Cullen, J. Virology, 62:673, 1988.

Hen et al., Nature, 321:249, 1986.

Hensel et al., Lymphokine Res., 8:347, 1989.

Herr and Clarke, Cell, 45:461, 1986.

Hirochika et al., J. Virol., 61:2599, 1987.

Hirsch et al., Mol. Cell. Biol., 10:1959, 1990.

Holbrook et al., Virology, 157:211, 1987.

Horlick and Benfield, Mol. Cell. Biol., 9:2396, 1989.

Huang et al., Cell, 27:245, 1981.

Hug H, Costas M, Staeheli P, Aebi M, et al. Organization of the murine Mx gene and characterization of its interferon- and virus-inducible promoter. Mol Cell Biol August 1988;8(8):3065-79.

Hwang et al., Mol. Cell. Biol., 10:585, 1990.

Imagawa et al., Cell, 51:251, 1987.

Imbra and Karin, Nature, 323:555, 1986.

Imler et al., Mol. Cell. Biol., 7:2558, 1987.

Jakobovits et al., Mol. Cell. Biol., 8:2555, 1988.

Jameel and Siddiqui, Mol. Cell. Biol., 6:710, 1986.

Jaynes et al., Mol. Cell. Biol., 8:62, 1988.

Johnson et al., Mol. Cell. Biol., 9:3393, 1989.

Kadesch and Berg, Mol. Cell. Biol., 6:2593, 1986.

Karin et al., Mol. Cell. Biol., 7:606, 1987.

Katinka et al., Cell, 20:393, 1980.

Katinka et al., Nature, 290:720, 1981.

Kawamoto et al., Mol. Cell. Biol., 8:267, 1988.

Kiledjian et al., Mol. Cell. Biol., 8:145, 1988.

Klamut et al., Mol. Cell. Biol., 10:193, 1990.

Koch et al., Mol. Cell. Biol., 9:303, 1989.

Kraus J, Woltje M, Schonwetter N, Hollt V. Alternative promoter usage and tissue specific expression of the mouse somatostatin receptor 2 gene. FEBS Lett May 29, 1998;428(3):165-70.

Kriegler and Botchan, In: Eukaryotic Viral Vectors, Y. Gluzman, ed., Cold Spring Harbor: Cold Spring Harbor Laboratory, N.Y., 1982.

Kriegler and Botchan, Mol. Cell. Biol., 3:325, 1983.

Kriegler et al., Cell, 38:483, 1984a.

Kriegler et al., Cell, 53:45, 1988.

Kriegler et al., In: Cancer Cells 2/Oncogenes and Viral Genes, Van de Woude et al. eds, Cold Spring Harbor, Cold Spring Harbor Laboratory, 1984b.

Kriegler et al., In: Gene Expression, D. Hamer and M. Rosenberg, eds., New York: Alan R. Liss, 1983.

Kuhl et al., Cell, 50:1057, 1987.

Kunz et al., Nucl. Acids Res., 17:1121, 1989.

Lareyre J J, Thomas T Z, Zheng W L, Kasper S, et al. A 5-kilobase pair promoter fragment of the murine epididymal retinoic acid-binding protein gene drives the tissue-specific, cell-specific, and androgen-regulated expression of a foreign gene in the epididymis of transgenic mice. J Biol Chem Mar. 19, 1999;274(12):8282-90.

Larsen et al., Proc. Nat'l Acad. Sci. USA., 83:8283, 1986.

Laspia et al., Cell, 59:283, 1989.

Latimer et al., Mol. Cell. Biol., 10:760, 1990.

Lee et al., Mol. Endocrinol., 2: 404-411, 1988.

Lee et al., Nature, 294:228, 1981.

Lee S H, Wang W, Yajima S, Jose P A, et al. Tissue-specific promoter usage in the D1A dopamine receptor gene in brain and kidney. DNA Cell Biol November 1997;16(11):1267-75.

Levinson et al., Nature, 295:79, 1982.

Li, X., Eastman, E. M., Schwartz, R. J., Draghia-Akli, R. Synthetic muscle promoters: activities exceeding naturally occurring regulatory sequences. nature biotechnology 17. 1999.

Lin et al., Mol. Cell. Biol., 10:850, 1990.

Lund, A. H.; Duch, M.; Pedersen, F. S. J. Biomed. Sci 3 (6): 365-378, 1996.

Luria et al., EMBO J., 6:3307, 1987.

Lusky and Botchan, Proc. Nat'l Acad. Sci. USA., 83:3609, 1986.

Lusky et al., Mol. Cell. Biol., 3:1108, 1983.

Majors and Varmus, Proc. Nat'l Acad. Sci. USA., 80:5866, 1983.

McNeall et al., Gene, 76:81, 1989.

Miksicek et al., Cell, 46:203, 1986.

Mir, L. M., Bureau, M. F., Gehl, J., Rangara, R., Rouy, D., Caillaud, J. M., Delaere, P., Branellec, D., Schwartz, B., Scherman, D. (1999) High-efficiency gene transfer into skeletal muscle mediated by electric pulses. Proc.Natl.Acad.Sci.U.S.A. 96, 4262-4267

Mir, L. M., Bureau, M. F., Rangara, R., Schwartz, B., Scherman, D. (1998) Long-term, high level in vivo gene expression after electric pulse-mediated gene transfer into skeletal muscle. C.R.Acad.Sci.III 321, 893-899

Mordacq and Linzer, Genes and Dev., 3:760, 1989.

Moreau et al., Nucl. Acids Res., 9:6047, 1981.

Muesing et al., Cell, 48:691, 1987.

Muramatsu, T., Nakamura, A., Park, H. M. (1998) In vivo electroporation: A powerful and convenient means of nonviral gene transfer to tissues of living animals (Review). Int.J.Mol.Med. 1, 55-62

Nairn, R. S., Adair, G. M. et al. (1993) Targeting vector configuration and method of gene transfer influence targeted correction of the APRT gene in Chinese hamster ovary cells. Som. Cell Mol. Genet. 19 (4):363-375

Neumann, E., Schaefer-Ridder, M., Wang, Y., Hofschneider, P. H. (1982) Gene transfer into mouse lyoma cells by electroporation in high electric fields. EMBO J. 1(7):841-845

Ng et al., Nuc. Acids Res., 17:601, 1989.

Nishi, T., Yoshizato, K., Yamashiro, S., Takeshima, H., Sato, K., Hamada, K., Kitamura, I., Yoshimura, T., Saya, H., Kuratsu, J., Ushio, Y. (1996) High-efficiency in vivo gene transfer using intraarterial plasmid DNA injection following in vivo electroporation. Cancer Res. 56, 1050-1055

Nomoto S, Tatematsu Y, Takahashi T, Osada H. Cloning and characterization of the alternative promoter regions of the human LIMK2 gene responsible for alternative transcripts with tissue-specific expression. Gene Aug. 20, 1999;236(2):259-71.

O'Hara, A. J., Howell, J. M., Taplin, R. H., Fletcher, S., Lloyd, F., Kakulas, B., Lochmuller, H., Karpati, G. (2001) The spread of transgene expression at the site of gene construct injection. Muscle Nerve 24, 488-495

Ondek et al., EMBO J., 6:1017, 1987.

Ornitz et al., Mol. Cell. Biol., 7:3466, 1987.

Palmiter et al., Nature, 300:611, 1982.

Pech et al., Mol. Cell. Biol., 9:396, 1989.

Perez-Stable and Constantini, Mol. Cell. Biol., 10:1116, 1990.

Picard and Schaffner, Nature, 307:83, 1984.

Pinkert et al., Genes and Dev., 1:268, 1987.

Ponta et al., Proc. Nat'l Acad. Sci. USA., 82:1020, 1985.

Porton et al., Mol. Cell. Biol., 10:1076, 1990.

Prentice, H., Kloner, R. A., Prigozy, T., Christensen, T., Newman, L., Li, Y., Kedes, L. (1994) Tissue restricted gene expression assayed by direct DNA injection into cardiac and skeletal muscle. Journal of Molecular & Cellular Cardiology 26, 1393-1401

Queen and Baltimore, Cell, 35:741, 1983.

Quinn et al., Mol. Cell. Biol., 9:4713, 1989.

Redondo et al., Science, 247:1225, 1990.

Reisman and Rotter, Mol. Cell. Biol., 9:3571, 1989.

Resendez Jr. et al., Mol. Cell. Biol., 8:4579, 1988.

Ripe et al., Mol. Cell. Biol., 9:2224, 1989.

Rittling et al., Nucl. Acids Res., 17:1619, 1989.

Rols, M. P., Delteil, C., Golzio, M., Dumond, P., Cros, S., Teissie, J. (1998) In vivo electrically mediated protein and gene transfer in murine melanoma. Nat.Biotechnol. 16, 168-171

Rosen et al., Cell, 41:813, 1988.

Satake et al., “Biological Activities of Oligonucleotides Spanning the F9 Point Mutation Within the Enhancer Region of Polyoma Virus DNA,” J. Virology, 62:970, 1988.

Schaffner et al., J. Mol. Biol., 201:81, 1988.

Searle et al., Mol. Cell. Biol., 5:1480, 1985.

Sharp and Marciniak, Cell, 59:229, 1989.

Shaul and Ben-Levy, EMBO J., 6:1913, 1987.

Sherman et al., Mol. Cell. Biol., 9:50, 1989.

Sleigh and Lockett, J. EMBO, 4:3831, 1985.

Shi, C. X.; Hitt, M.; Ng, P.; Graham, F. L. (2002) Hum. Gene Thr. 13 (2), 211-224

Smith, L. C., Nordstrom, J. L. (2000) Advances in plasmid gene delivery and expression in skeletal muscle. Curr.Opin.Mol.Ther. 2, 150-154

Soubrier, F., Cameron, B., Manse, B., Somarriba, S., Dubertret, C., Jaslin, G., Jung, G., Caer, C. L., Dang, D., Mouvault, J. M., Scherman, D., Mayaux, J. F., Crouzet, J. (1999) pCOR: a new design of plasmid vectors for nonviral gene therapy. Gene Ther. 6, 1482-1488

Spalholz et al., Cell, 42:183, 1985.

Spandau and Lee, J. Virology, 62:427, 1988.

Spandidos and Wilkie, EMBO J., 2:1193, 1983.

Stan, A. C., Casares, S., Brumeanu, T. D., Klinman, D. M., Bona, C. A. (2001) CpG motifs of DNA vaccines induce the expression of chemokines and MHC class II molecules on myocytes. Eur.J Immunol. 31, 301-310

Stephens and Hentschel, Biochem. J., 248:1, 1987.

Stuart et al., Nature, 317:828, 1985.

Sullivan and Peterlin, Mol. Cell. Biol., 7:3315, 1987.

Swartzendruber and Lehman, J. Cell. Physiology, 85:179, 1975.

Takebe et al., Mol. Cell. Biol., 8:466, 1988.

Tavernier et al., Nature, 301:634, 1983.

Taylor and Kingston, Mol. Cell. Biol., 10:165, 1990a.

Taylor and Kingston, Mol. Cell. Biol., 10:176, 1990b.

Taylor et al., J. Biol. Chem., 264:15160, 1989.

Thiesen et al., J. Virology, 62:614, 1988.

Toneguzzo, F., Keating, A., Glynn, S., McDonald, K. (1988) Electric field-mediated gene transfer: characterization of DNA transfer and patterns of integration in lymphoid cells. Nucl. Acids Res. 16(12):5515-5532

Treisman, Cell, 42:889, 1985.

Tronche et al., Mol. Biol. Med., 7:173, 1990.

Tronche et al., Mol. Cell. Biol., 9:4759, 1989.

Trudel and Constantini, Genes and Dev., 6:954, 1987.

Tsumaki N, Kimura T, Tanaka K, Kimura J H, et al. Modular arrangement of cartilage- and neural tissue-specific cis-elements in the mouse alpha2(XI) collagen promoter. J Biol Chem Sep. 4, 1998;273(36):22861-4.

Tyndall et al., Nuc. Acids. Res., 9:6231, 1981.

Vannice and Levinson, J. Virology, 62:1305, 1988.

Vasseur et al., Proc. Nat'l Acad. Sci. USA., 77:1068, 1980.

Wang and Calame, Cell, 47:241, 1986.

Wang, Y.; Camp, S. M.; Niwano, M.; Shen, X.; Bakowska, J. C.; Breakefield, X. O.; Allen, P. D. (2002) J. Virol. 76 (14): 7150-7162

Weber et al., Cell, 36:983, 1984.

Weinberger et al. Mol. Cell. Biol., 8:988, 1984.

Wells, K. E., Maule, J., Kingston, R., Foster, K., McMahon, J., Damien, E., Poole, A., Wells, D. J. (1997) Immune responses, not promoter inactivation, are responsible for decreased long-term expression following plasmid gene transfer into skeletal muscle. FEBS Lett. 407, 164-168

Winoto and Baltimore, Cell, 59:649, 1989.

Wolff, J. A., Malone, R. W., Williams, P., Chong, W., Acsadi, G., Jani, A., Feigner, P L. (1990) Direct gene transfer into mouse muscle in vivo. Science 247, 1465-1468

Wu H K, Squire J A, Song Q, Weksberg R. Promoter-dependent tissue-specific expressive nature of imprinting gene, insulin-like growth factor II, in human tissues. Biochem Biophys Res Commun Apr 7, 1997; 233(1):221-6.

Xie, T.-D. and Tsong, T. Y. (1993) Study of mechanisms of electric field-induced DNA transfection. V. Effects of DNA topology on surface binding, cell uptake, expression and integration into host chromosomes of DNA in the mammalian cell. Biophys. J. 65:1684-1689

Yew, N. S., Zhao, H., Wu, I. H., Song, A., Tousignant, J. D., Przybylska, M., Cheng, S. H. (2000) Reduced inflammatory response to plasmid DNA vectors by elimination and inhibition of immunostimulatory CpG motifs. Mol.Ther. 1, 255-262

Yorifuji, T. and Mikawa, H. (1990) Co-transfer of restriction endonucleases and plasmid DNA into mammalian cells by electroporation: effects on stable transformation. Mut. Res. 243:121-126

Yutzey et al. “An Internal Regulatory Element Controls Troponin I Gene Expression,” Mol. Cell. Biol., 9:1397, 1989.

Yutzey et al. Mol. Cell. Biol., 9:1397, 1989.

Zhao-Emonet J C, Boyer O, Cohen J L, Klatzmann D. Deletional and mutational analyses of the human CD4 gene promoter: characterization of a minimal tissue-specific promoter. Biochem Biophys Acta Nov. 8, 1998; 1442(2-3):109-19.

What is claimed is:

1. A construct for plasmid mediated gene supplementation, the construct being a linear double-stranded nucleic acid expression plasmid comprising:
(a) a promoter;
(b) a nucleotide sequence of interest; and
(c) a 3′ untranslated region;
wherein:
the construct is substantially free from a viral backbone;
the promoter, the nucleotide sequence of interest, and the 3′ untranslated region are operably linked; and
in vivo expression of the nucleotide sequence of interest is regulated by the promoter.

2. The construct of claim 1, further comprising a residual linear plasmid backbone, wherein the linear plasmid backbone is substantially free of viral backbone.

3. The construct of claim 1, wherein the nucleotide sequence of interest encodes a hormone or an enzyme.

4. The construct of claim 3, wherein the hormone comprises growth hormone releasing hormone.

5. The construct of claim 3, wherein the hormone is growth hormone, insulin, glucagon, adrenocorticotropic hormone, thyroid stimulating hormone, follicle-stimulating hormone, insulin growth factor I, insulin growth factor II, corticotropin-releasing hormone, parathyroid hormone, calcitonin, chorionic gonadotropin, luteinizing hormone, chorionic somatomammotropin, cholecystokinin, secretin, prolactin, oxytocin, vasopressin, angiotensin, melanocyte-stimulating hormone, somatostatin, thyrotropin-releasing hormone, gonadotropin-releasing hormone, or gastrin.

6. The construct of claim 3, wherein the enzyme is secreted embryonic alkaline phosphatase, glucuronidase, arylsulfatase, factor VIII, factor IX, or beta-galactosidase.

7. The construct of claim 1, wherein the nucleotide sequence of interest encodes a cytokine.

8. The construct of claim 7, wherein the cytokine is IL-2 or IL-7.

9. The construct of claim 1, wherein the promoter comprises a tissue-specific promoter.

10. The construct of claim 9, wherein the tissue-specific promoter comprises a muscle-specific promoter.

11. The construct of claim 1, wherein the promoter comprises SPc5-12.

12. The construct of claim 1, wherein the 3′ untranslated region is human growth hormone 3′ UTR, bovine growth hormone 3′ UTR, skeletal alpha actin 3′ UTR, or SV40 polyadenylation signal.

13. A method for increasing levels of a polypeptide in a subject comprising the steps of:
(a) delivering a linear double stranded nucleic acid expression construct into a selected tissue; and
(b) applying a cell-transfecting pulse to the selected tissue;
wherein:
the construct is substantially free from a viral backbone;
the polypeptide is encoded by a gene sequence on the linear double-stranded nucleic acid expression construct; and
the linear double-stranded nucleic acid expression construct is delivered in an area comprising the cell-transfecting pulse.

14. The method of claim 13, wherein the linear double-stranded nucleic acid expression construct comprising:
(a) a promoter;
(b) a nucleotide sequence of interest; and
(c) a 3′ untranslated region;
wherein the promoter, the nucleotide sequence of interest, and the 3′ untranslated region are operably linked; and
in vivo expression of the nucleotide sequence of interest is regulated by the promoter.

15. The construct of claim 14, further comprising a residual linear plasmid backbone, wherein the linear plasmid backbone is substantially free of viral backbone.

16. The method of claim 14, wherein the nucleotide sequence of interest encodes a hormone or an enzyme.

17. The method of claim 16, wherein the hormone comprises growth hormone releasing hormone.

18. The method of claim 17, wherein the hormone is growth hormone, insulin, glucagon, adrenocorticotropic hormone, thyroid stimulating hormone, follicle-stimulating hormone, insulin growth factor I, insulin growth factor II, corticotropin-releasing hormone, parathyroid hormone, calcitonin, chorionic gonadotropin, luteinizing hormone, chorionic somatomammotropin, cholecystokinin, secretin, prolactin, oxytocin, vasopressin, angiotensin, melanocyte-stimulating hormone, somatostatin, thyrotropin-releasing hormone, gonadotropin-releasing hormone, or gastrin.

19. The method of claim 17, wherein the enzyme is secreted embryonic alkaline phosphatase, glucuronidase, arylsulfatase, factor VIII, factor IX, or beta-galactosidase.

20. The method of claim 14, wherein the nucleotide sequence of interest encodes a cytokine.

21. The method of claim 20, wherein the cytokine is IL-2 or IL-7.

22. The method of claim 14, wherein the promoter comprises a tissue-specific promoter.

23. The method of claim 22, wherein the tissue-specific promoter comprises a muscle-specific promoter.

24. The construct of claim 14, wherein the promoter comprises SPc5-12.

25. The method of claim 14, wherein the 3′ untranslated region is human growth hormone 3′ UTR, bovine growth hormone 3′ UTR, skeletal alpha actin 3′ UTR, or SV40 polyadenylation signal.

26. The method of claim 13, wherein the delivering step is by injection, gene gun, or gold particle bombardment.

27. The method of claim 13, wherein the tissue comprises muscle.

28. The method of claim 13, wherein the subject is a human, a pig, a horse, a cow, a mouse, a rat, a monkey, a sheep, a goat, a dog, or a cat.

29. The method of claim 13, further comprising placing a plurality of electrodes in the selected tissue before applying the cell-transfecting pulse to the selected tissue, wherein the linear double stranded nucleic acid expression construct is delivered to the selected tissue in an area that interposes the plurality of electrodes.

30. The method of claim 29, wherein the cell-transfecting pulse comprises an electrical pulse.

31. The method of claim 13, wherein the cell-transfecting pulse is an electrical pulse or a vascular pressure pulse.

32. A method for increasing levels of a polypeptide in a subject comprising the steps of:
(a) placing a plurality of electrodes in the selected tissue,
(b) delivering the linear double stranded nucleic acid expression construct into the selected tissue; and
(c) applying an electrical pulse to the plurality of electrodes;
wherein:
the construct is substantially free from a viral backbone;
the polypeptide is encoded by a gene sequence on the linear double-stranded nucleic acid expression construct; and
the linear double stranded nucleic acid expression construct is delivered to the selected tissue in an area that interposes the plurality of electrodes.

33. The method of claim 32, wherein the linear double-stranded nucleic acid expression construct comprising:
(a) a promoter;
(b) a nucleotide sequence of interest; and
(c) a 3′ untranslated region;
wherein the promoter, the nucleotide sequence of interest, and the 3′ untranslated region are operably linked; and
in vivo expression of the nucleotide sequence of interest is regulated by the promoter.

34. The construct of claim 33, further comprising a residual linear plasmid backbone, wherein the residual linear plasmid backbone is substantially free of viral backbone.

35. The method of claim 33, wherein the nucleotide sequence of interest encodes a hormone or an enzyme.

36. The method of claim 35, wherein the hormone comprises growth hormone releasing hormone.

37. The method of claim 36, wherein the hormone is growth hormone, insulin, glucagon, adrenocorticotropic hormone, thyroid stimulating hormone, follicle-stimulating hormone, insulin growth factor I, insulin growth factor II, corticotropin-releasing hormone, parathyroid hormone, calcitonin, chorionic gonadotropin, luteinizing hormone, chorionic somatomammotropin, cholecystokinin, secretin, prolactin, oxytocin, vasopressin, angiotensin, melanocyte-stimulating hormone, somatostatin, thyrotropin-releasing hormone, gonadotropin-releasing hormone, or gastrin.

38. The method of claim 36, wherein the enzyme is secreted embryonic alkaline phosphatase, glucuronidase, arylsulfatase, factor VIII, factor IX, or beta-galactosidase.

39. The method of claim 33, wherein the nucleotide sequence of interest encodes a cytokine.

40. The method of claim 39, wherein the cytokine is IL-2 or IL-7.

41. The method of claim 33, wherein the promoter comprises a tissue-specific promoter.

42. The method of claim 41, wherein the tissue-specific promoter comprises a muscle-specific promoter.

43. The construct of claim 33, wherein the promoter comprises SPc5-12.

44. The method of claim 33, wherein the 3′ untranslated region is human growth hormone 3′ UTR, bovine growth hormone 3′ UTR, skeletal alpha actin 3′ UTR, or SV40 polyadenylation signal.

45. The method of claim 32, wherein the delivering step is by injection, gene gun, or gold particle bombardment.

46. The method of claim 32, wherein the tissue comprises muscle.

47. The method of claim 32, wherein the subject is a human, a pig, a horse, a cow, a mouse, a rat, a monkey, a sheep, a goat, a dog, or a cat.

48. A construct for plasmid mediated gene supplementation, the construct being a linear double-stranded nucleic acid expression plasmid comprising:
(a) a promoter;
(b) a nucleotide sequence of interest; and
(c) a 3′ untranslated region;
wherein:
the construct is substantially free from a viral backbone;
the promoter, the nucleotide sequence of interest, and the 3′ untranslated region are operably linked; and
the nucleotide sequence of interest comprises a growth hormone releasing hormone;
the promoter comprises a tissue-specific promoter;
the 3′ untranslated region comprises a human growth hormone 3′ UTR;
in vivo expression of the nucleotide sequence of interest is regulated by the promoter.

49. A method for increasing levels of a polypeptide in a subject comprising the steps of:
(a) placing a plurality of electrodes in the selected tissue,
(b) delivering the linear double stranded nucleic acid expression construct into the selected tissue; and
(c) applying an electrical pulse to the plurality of electrodes;
wherein the polypeptide is encoded by a gene sequence on the linear double-stranded nucleic acid expression construct; the linear double-stranded nucleic acid expression construct comprising:
a promoter;
a nucleotide sequence of interest;
a 3′ untranslated region; and
the promoter, the nucleotide sequence of interest, and the 3′ untranslated region are operably linked;
the nucleotide sequence of interest comprises a growth hormone releasing hormone;
the promoter comprises a tissue-specific promoter;
the 3′ untranslated region comprises a human growth hormone 3′ UTR;
and in vivo expression of the nucleotide sequence of interest is regulated by the promoter;
the construct being substantially free from a viral backbone;
the linear double stranded nucleic acid expression construct is delivered to the selected tissue in an area that interposes the plurality of electrodes;
the delivering step comprises injection; and
the tissue comprises muscle.
